(19) United States
(12) Patent Application Publication
(10) Pub. No.: US 2025/0295849 A1
(43) Pub. Date: Sep. 25, 2025

(54) IRRIGATOR COMPONENT AND NASAL IRRIGATOR

(71) Applicant: Medcodes Healthcare (Shenzhen) Limited, Shenzhen (CN)

(72) Inventors: Shengqiang DAI, Shenzhen (CN); Chuanzhou ZHENG, Shenzhen (CN); Dongbao LI, Shenzhen (CN)

(73) Assignee: Medcodes Healthcare (Shenzhen) Limited, Shenzhen (CN)

(21) Appl. No.: 18/816,255

(22) Filed: Aug. 27, 2024

(30) Foreign Application Priority Data
Mar. 19, 2024 (CN) 202420561634.3

(51) Int. Cl. A61M 3/02

(52) U.S. Cl. CPC A61M 3/0283; A61M 3/0258; A61M 2210/0618

(57) ABSTRACT

Disclosed are an irrigator component and a nasal irrigator. The irrigator component includes: a body, a water tank, a water tank, a first nozzle and a second nozzle. The body includes a first connection portion and a second connection portion; the water tank is configured to store cleaning liquid, and the water tank is connected to the first connection portion; the waste tank is configured to recycle the cleaning liquid, and the waste tank is connected to the second connection portion; and the first nozzle is communicated with the water tank and the second nozzle is communicated with the waste tank. The cleaning liquid is configured to enter user's nasal cavity through the first nozzle, and flow back to the waste tank from the second nozzle.

### CROSS-REFERENCE TO RELATED APPLICATIONS

The present application claims priority to Chinese Patent Application No. 202420561634.3, filed on Mar. 19, 2024, the entire contents of which are incorporated herein by reference.

### TECHNICAL FIELD

The present application relates to the technical field of medical devices, and in particular to an irrigator component and a nasal irrigator.

### BACKGROUND

The nasal irrigator is a tool for cleaning the nasal cavity, which uses a water pump to introduce the liquid in the water tank into the nozzle, and the nozzle sends the liquid into one nostril. The liquid flows through the nasal vestibule, sinuses, nasal passages, bypasses the nasopharynx, and is discharged from the other nostril, or discharged from the mouth. Through the above path, with the help of the impact force of the physiological saline water flow, the pathogenic bacteria and dirt that have accumulated in the nasal cavity are discharged, so that the nasal cavity restores the normal physiological environment, restores the self-detoxification function of the nasal cavity, and achieves the purpose of protecting the nasal cavity.

In the use process of the existing nasal irrigator, since the liquid is directly discharged from the nostril or the mouth, the discharged liquid falls into the pool or other places and is easy to splash, and the liquid cannot be collected, resulting in the user's clothes or the external environment being wet, thereby affecting the user's experience.

### SUMMARY

The main purpose of the present application is to provide an irrigator component, aiming to solve the problem that the existing nasal irrigator cannot recycle cleaning liquid.

To achieve the above purpose, the present application provides an irrigator component, including:
a body including a first connection portion and a second connection portion;
a water tank configured to store cleaning liquid, the water tank is connected to the first connection portion;
a waste tank configured to recycle the cleaning liquid, the waste tank is connected to the second connection portion; and
a first nozzle communicated with the water tank and a second nozzle communicated with the waste tank, cleaning liquid is configured to enter user's nasal cavity through the first nozzle, and flow back to the waste tank from the second nozzle.

In an embodiment, the first connection portion is provided at one end of the body close to the water tank, and the first connection portion includes a first abutting section and a second abutting section connected to the first abutting section; and
the first abutting section and the second abutting section are enclosed to form a clamping groove; a clamping protrusion is provided in the water tank, and the clamping protrusion is clamped in the clamping groove.

In an embodiment, one end of the first abutting section is connected to the second abutting section, and the clamping groove is provided with an opening;
the opening is provided at one end of the first abutting section away from the second abutting section; and
one end of the clamping protrusion is configured to enter the clamping groove from the opening, and move towards a direction close to the second abutting section to be clamped in the clamping groove.

In an embodiment, the irrigator component further includes:
a pipe assembly and a water pump provided in the body,
the pipe assembly includes a first pipe communicated with the first nozzle and the water tank and a second pipe communicated with the second nozzle and the waste tank; and
the water pump is configured to drive the cleaning liquid into the first nozzle.

In an embodiment, the pipe assembly further includes a third pipe communicated with the water tank and the waste tank, so that air pressure in the water tank is equal to air pressure in the waste tank.

In an embodiment, the water tank is provided with a liquid inlet for introducing the cleaning liquid, and the liquid inlet is provided at one end of the water tank away from the first connection portion; and
the water tank further includes a waterproof cover snapped onto the liquid inlet and covering the liquid inlet.

In an embodiment, the waterproof cover includes a waterproof portion and a connection column;
one end of the connection column is connected to the waterproof portion, and the other end of the connection column is provided with an anti-release portion; and
the connection column is configured to pass through the water tank and the anti-release portion is provided in the water tank.

In an embodiment, the water tank further includes a seal member, and an inserting portion for engaging with the water tank is provided on one side of the waterproof portion close to the connection column; and
the inserting portion is provided with an annular groove, and the seal member is partially provided in the annular groove and abutted against the water tank.

In an embodiment, the first nozzle is inclined at a certain angle towards a direction close to the second nozzle, and the second nozzle is inclined at a same angle towards a direction close to the first nozzle.

The present application also provides a nasal irrigator, including the irrigator component.

In the technical solution of the present application, two nozzles are connected to the water tank and the waste tank respectively. Cleaning liquid is introduced from the water tank into the first nose of the user through the first nozzle, and flows back to the waste tank through the second nozzle in the second nose of the user, thereby avoiding cleaning liquid being directly discharged from the nose or mouth and splashing.

### BRIEF DESCRIPTION OF THE DRAWINGS

In order to illustrate the technical solutions in the embodiments of the present application or in the related art more clearly, the following briefly introduces the accompanying drawings required for the description of the embodiments or the related art. Obviously, the drawings in the following description are only part of embodiments of the present application. For those skilled in the art, other drawings can also be obtained according to the structures shown in these drawings without any creative effort.

FIG. 1 is a schematic structural view of a nasal irrigator according to an embodiment of the present application.

FIG. 2 is a schematic view of an irrigator component according to an embodiment of the present application.

FIG. 3 is a schematic view of the irrigator component according to an embodiment of the present application.

FIG. 4 is an enlarged view at A in FIG. 3.

FIG. 5 is a schematic view of the irrigator component according to an embodiment of the present application.

FIG. 6 is a schematic structural view of the nasal irrigator according to an embodiment of the present application.

### DETAILED DESCRIPTION OF THE EMBODIMENTS

The technical solutions of the embodiments of the present application will be described in more detail below with reference to the accompanying drawings. It is obvious that the embodiments to be described are only some rather than all of the embodiments of the present application. All other embodiments obtained by those skilled in the art based on the embodiments of the present application without creative efforts shall fall within the scope of the present application.

It should be noted that if there are directional indications, such as up, down, left, right, front, back, etc., involved in the embodiments of the present application, the directional indications are only used to explain a certain posture as shown in the accompanying drawings. If the specific posture changes, the directional indication also changes accordingly.

In addition, if there are descriptions related to “first”, “second”, etc. in the embodiments of the present application, the descriptions of “first”, “second”, etc. are only for the purpose of description, and should not be construed as indicating or implying relative importance or implicitly indicates the number of technical features indicated. Thus, a feature delimited with “first”, “second” may expressly or implicitly include at least one of that feature. In addition, the technical solutions between the various embodiments can be combined with each other, but must be based on the realization by those skilled in the art. When the combination of technical solutions is contradictory or cannot be realized, it should be considered that the combination of such technical solutions does not exist or fall within the scope of protection claimed in the present application.

The nasal irrigator is a tool for cleaning the nasal cavity, which uses a water pump to introduce the liquid in the water tank into the nozzle, and the nozzle sends the liquid into one nostril. The liquid flows through the nasal vestibule, sinuses, nasal passages, bypasses the nasopharynx, and is discharged from the other nostril or from the mouth. Through the above path, with the help of the impact force of the saline water flow, the pathogenic bacteria and dirt that have accumulated in the nasal cavity are discharged, so that the nasal cavity can restore its normal physiological environment, restore the self-detoxification function of the nasal cavity, and achieve the purpose of protecting the nasal cavity.

In the use process of the existing nasal irrigator, since the liquid is discharged directly from the nostril or mouth, the discharged liquid is easy to splash into the pool or other places, and the liquid cannot be collected, resulting in the user's clothes or the external environment being wet, thereby affecting the user's experience.

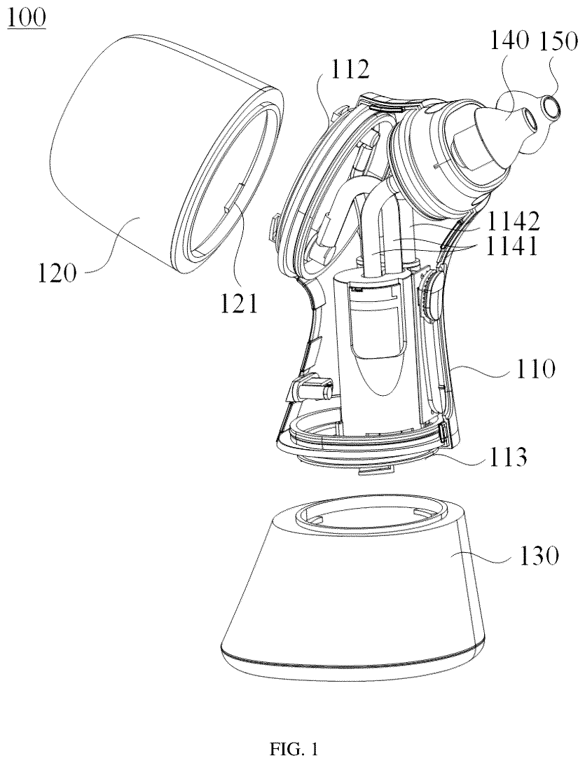

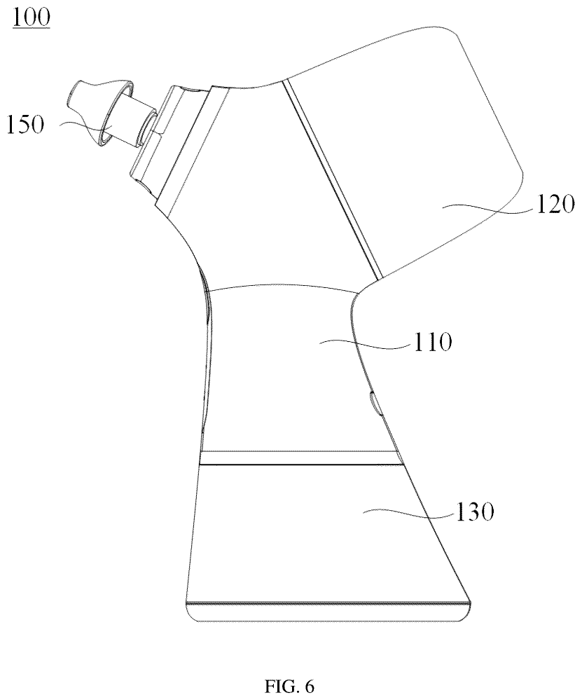

In order to solve the above problems, referring to FIG. 1 and FIG. 6, the present application provides an irrigator component, which includes a body 110, a water tank 120, a waste tank 130, and a first nozzle 140 and a second nozzle 150.

The body 110 includes a first connection portion 112 and a second connection portion 113; the water tank 120 is configured to store cleaning liquid, and the water tank 120 is connected to the first connection portion 112; the waste tank 130 is configured to recycle cleaning liquid, and the waste tank 130 is connected to the second connection portion 113; and

The first nozzle 140 is connected to the water tank 120, the second nozzle 150 is connected to the waste tank 130, and cleaning liquid enters the nasal cavity of the user through the first nozzle 140 and flows back to the waste tank 130 from the second nozzle 150.

In this embodiment, two nozzles are respectively connected to the water tank 120 and the waste tank 130. cleaning liquid is introduced from the water tank 120 into the user's first nostril through the first nozzle 140, and flows back to the waste tank 130 through the second nozzle 150 in the user's second nostril, thereby avoiding cleaning liquid being directly discharged from the nostril or mouth and splashing.

Specifically, when the water tank 120 and the waste tank 130 are connected to the body 110, the nasal irrigator 100 is in a “Y” shape as a whole. Specifically, referring to the positional relationship in FIG. 6, the waste tank 130 is connected to the bottom of the body 110, the water tank 120 is connected to the upper right of the body 110, and the first nozzle 140 and the second nozzle 150 are both connected to the upper left of the body 110. When the nasal irrigator 100 is used, the palm of the user holds the body 110 relative to the body 110, and the water tank 120 is abutted against the upper part of the palm, so that the user can conveniently take and use the nasal irrigator 100. At the same time, the overall “Y” shape of the nasal irrigator 100 improves the aesthetics of the nasal irrigator 100.

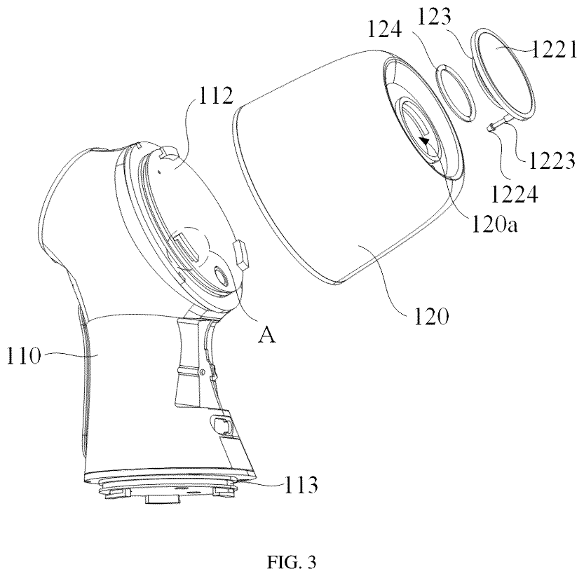

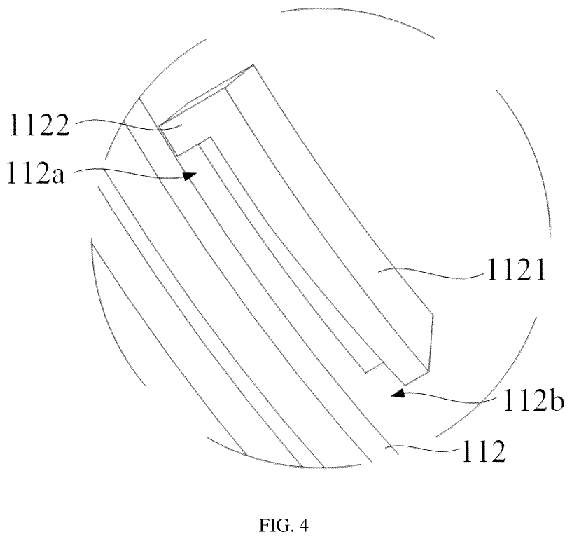

In an embodiment, referring to FIG. 3 and FIG. 4, the first connection portion 112 is provided at one end of the body 110 close to the water tank 120. The first connection portion 112 includes a first abutting section 1121 and a second abutting section 1122 connected to the first abutting section 1121, and the first abutting section 1121 and the second abutting section 1122 enclose a clamping groove 112a. A clamping protrusion 121 is provided in the water tank 120, and the clamping protrusion 121 is clamped in the clamping groove 112a.

In this embodiment, in order to facilitate the disassembly and installation of the water tank 120 and to facilitate the cleaning of the water tank 120, the connection method of the water tank 120 and the body 110 adopts the matching method of the clamping protrusion 121 and the clamping groove 112a. In other embodiments, the connection method of the water tank 120 and the body 110 can be other methods such as threaded connection, which is not limited here.

In an embodiment, referring to FIG. 4, one end of the first abutting section 1121 is connected to the second abutting section 1122. The clamping groove 112a is provided with an opening 112b, and the opening 112b is provided at one end of the first abutting section 1121 away from the second abutting section 1122. One end of the clamping protrusion 121 enters the clamping groove 112a from the opening 112b, and moves towards the direction close to the second abutting section 1122 to be clamped in the clamping groove 112a.

In this embodiment, the first clamping portion and the water tank 120 are uniformly cylindrical, and the clamping protrusion 121 is extended along the circumferential direction of the water tank 120; the first abutting section 1121 is extended along the circumferential direction of the first clamping portion, and the second abutting section 1122 is perpendicular to the first abutting section 1121. When the clamping protrusion 121 is clamped in the clamping groove 112a, one end of the clamping protrusion 121 is abutted against the second abutting section 1122, and one side of the clamping protrusion 121 is abutted against the first abutting section 1121, thereby limiting the water tank 120 in the circumferential direction and the axial direction and preventing the water tank 120 from moving in the circumferential direction and axial direction relative to the first clamping portion.

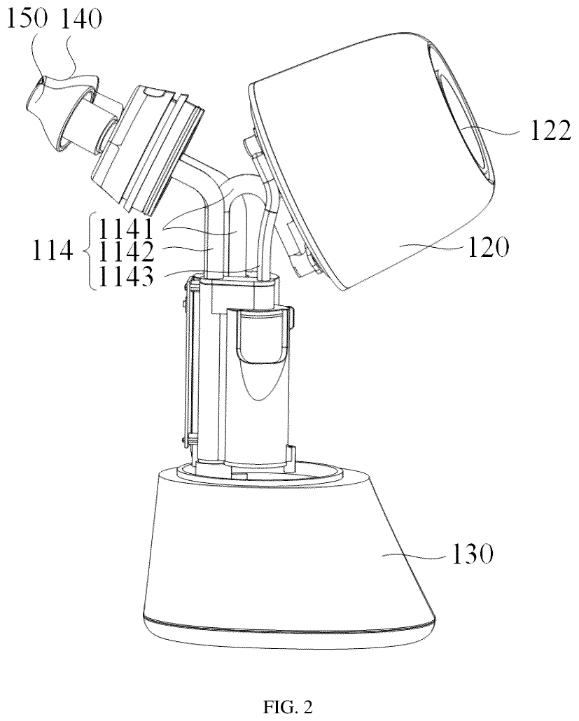

In an embodiment, referring to FIG. 1 and FIG. 2, the irrigator component further includes a pipe assembly 114 and a water pump provided in the body 110. The pipe assembly 114 includes a first pipe 1141 and a second pipe 1142. The first pipe 1141 is communicated with the first nozzle 140 and the water tank 120, and the second pipe 1142 is communicated with the second nozzle 150 and the waste tank 130. The water pump is configured to drive cleaning liquid into the first nozzle 140.

In this embodiment, the pipe assembly 114 is provided to guide the flow direction of cleaning liquid, so as to prevent cleaning liquid from flowing into the core component in the body 110, otherwise causing the core component to be short-circuited due to contamination of cleaning liquid. At the same time, a water pump (not shown in the figure) is provided to ensure that cleaning liquid can flow from the water tank 120 to the first nozzle 140 and enter the nasal cavity of the user. The present application does not provide a switch valve in the first pipe 1141 to control cleaning liquid to be sprayed out from the first nozzle 140, but directly drives cleaning liquid from the water tank 120 to the first nozzle 140 through the water pump, thereby simplifying the structure of the present application and reducing the production cost.

In an embodiment, referring to FIG. 1 and FIG. 2, the pipe assembly 114 also includes a third pipe 1143, communicated with the water tank 120 and the waste tank 130 to make the air pressure in the water tank 120 and the waste tank 130 equal.

In this embodiment, since the volume of cleaning liquid in the water tank 120 decreases with the use of the nasal irrigator 100, and the volume of the gas in the water tank 120 increases, resulting in a decrease in pressure. Similarly, the volume of cleaning liquid in the waste tank 130 continues to increase with the use of the nasal irrigator 100, the volume of gas continues to decrease, and the pressure increases. The pressure difference between the water tank 120 and the waste tank 130 makes it difficult for cleaning liquid to flow from the water tank 120 to the waste tank 130. Therefore, the third pipe 1143 is communicated with the water tank 120 and the waste tank 130, so that the air pressures therebetween are equal, thereby ensuring the normal use of the nasal irrigator 100.

In an embodiment, referring to FIG. 3, the water tank 120 is provided with a liquid inlet 120a for introducing cleaning liquid, and the liquid inlet 120a is provided at one end of the water tank 120 away from the first connection portion 112. The water tank 120 also includes a waterproof cover 122 snapped on the liquid inlet 120a and covers the liquid inlet 120a.

In this embodiment, in order to facilitate adding cleaning liquid, the liquid inlet 120a is provided at the water tank 120, and the waterproof cover 122 is provided at the liquid inlet 120a to prevent cleaning liquid in the water tank 120 from flowing out from the liquid inlet 120a during the use of the nasal irrigator 100, thereby improving the sealing of the water tank 120.

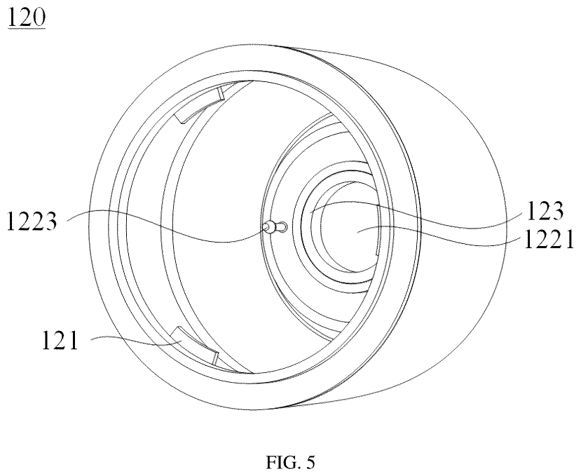

In an embodiment, referring to FIG. 3 and FIG. 5, the waterproof cover 122 includes a waterproof portion 1221 and a connection column 1223. One end of the connection column 1223 is connected to the waterproof portion 1221, and the other end of the connection column 1223 is provided with an anti-release portion 1224. The connection column 1223 is passed through the water tank 120, and the anti-release portion 1224 is provided in the water tank 120.

In this embodiment, the connection column 1223 is provided in the water tank 120. One end of the connection column 1223 is connected to the waterproof portion 1221 of the waterproof cover 122, and the other end of the connection column 1223 is provided with the anti-release portion 1224. The anti-release portion 1224 is provided in the water tank 120 and abutted against the inner wall of the water tank 120. When the waterproof portion 1221 is away from the liquid inlet 120a, that is, the waterproof cover 122 does not cover the liquid inlet 120a, the waterproof cover 122 is still connected to the water tank 120, so as to avoid the situation where the waterproof cover 122 has nowhere to be placed when cleaning liquid needs to be introduced into the liquid inlet 120a.

In an embodiment, referring to FIG. 3, the water tank 120 further includes a seal member 124. The waterproof portion 1221 is provided with an inserting portion 123 for engaging with the water tank 120 on one side close to the connection column 1223, and the inserting portion 123 is provided with an annular groove. The seal member 124 is partially provided in the annular groove and abutted against the water tank 120.

In this embodiment, in order to improve the waterproofness of the water tank 120, the annular groove is provided in the inserting portion 123, and the seal member 124 is placed in the annular groove to prevent cleaning liquid in the water tank 120 from flowing out from the gap between the inserting portion 123 and the water tank 120.

In an embodiment, referring to FIG. 1, the first nozzle 140 is inclined at a certain angle towards the direction close to the second nozzle 150, and the second nozzle 150 is inclined at the same angle towards the direction close to the first nozzle 140.

In this embodiment, in order to facilitate the user to insert the first nozzle 140 and the second nozzle 150 into the nose, the first nozzle 140 and the second nozzle 150 are inclined at an angle of 15° to 30°, which makes the nozzle fitted with the nose of the user better and prevents cleaning liquid from rushing out from the gap between the nose and the outer wall of the nozzle. In an embodiment, the first nozzle 140 and the second nozzle 150 are made of a silicone material with good elasticity to reduce the foreign body sensation in the nasal cavity of the user and improve the user's experience.

The present application also provides a nasal irrigator 100, including the irrigator component. The specific structure of the irrigator component refers to the above embodiment. Since the present nasal irrigator 100 adopts all the technical solutions of all the above embodiments, it at least has all the beneficial effects brought by the technical solutions of the above embodiments, which will not be repeated here.

The above descriptions are only embodiments of the present application, and are not intended to limit the scope of the present application. Under the inventive concept of the present application, any equivalent structural transformations made by using the contents of the description and drawings of the present application, or direct/indirect applications in other related technical fields are included in the scope of the present application.

1. An irrigator component, comprising:
a body comprising a first connection portion and a second connection portion;
a water tank configured to store cleaning liquid, wherein the water tank is connected to the first connection portion;
a waste tank configured to recycle the cleaning liquid, wherein the waste tank is connected to the second connection portion;
a first nozzle communicated with the water tank and a second nozzle communicated with the waste tank, wherein the cleaning liquid is configured to enter a user's nasal cavity through the first nozzle, and flow back to the waste tank from the second nozzle; and
a pipe assembly and a water pump provided in the body, wherein the pipe assembly comprises a first pipe communicated with the first nozzle and the water tank and a second pipe communicated with the second nozzle and the waste tank; the water pump is configured to drive the cleaning liquid into the first nozzle; and the pipe assembly further comprises a third pipe communicated with the water tank and the waste tank, so that air pressure in the water tank is equal to air pressure in the waste tank.

2. The irrigator component according to claim 1, wherein the first connection portion is provided at one end of the body close to the water tank, and the first connection portion comprises a first abutting section and a second abutting section connected to the first abutting section; and
the first abutting section and the second abutting section are enclosed to form a clamping groove; a clamping protrusion is provided in the water tank, and the clamping protrusion is clamped in the clamping groove.

3. The irrigator component according to claim 2, wherein one end of the first abutting section is connected to the second abutting section, and the clamping groove is provided with an opening;
the opening is provided at one end of the first abutting section away from the second abutting section; and
one end of the clamping protrusion is configured to enter the clamping groove from the opening, and move towards a direction close to the second abutting section to be clamped in the clamping groove.

4-5. (canceled)

6. The irrigator component according to claim 1, wherein the water tank is provided with a liquid inlet for introducing the cleaning liquid, and the liquid inlet is provided at one end of the water tank away from the first connection portion; and
the water tank further comprises a waterproof cover snapped onto the liquid inlet and covering the liquid inlet.

7. The irrigator component according to claim 6, wherein the waterproof cover comprises a waterproof portion and a connection column;
one end of the connection column is connected to the waterproof portion, and the other end of the connection column is provided with an anti-release portion; and
the connection column is configured to pass through the water tank and the anti-release portion is provided in the water tank.

8. The irrigator component according to claim 7, wherein the water tank further comprises a seal member, and an inserting portion for engaging with the water tank is provided on one side of the waterproof portion close to the connection column; and
the inserting portion is provided with an annular groove, and the seal member is partially provided in the annular groove and abutted against the water tank.

9. The irrigator component according to claim 1, wherein the first nozzle is inclined at an angle of 15° to 30° towards the second nozzle, and the second nozzle is inclined at a same angle towards the first nozzle.

10. A nasal irrigator, comprising the irrigator component according to claim 1.
